SEQUENCE LISTING (1 sequence)

SEQ ID NO: 1 - protein, 639 aa, synthetic construct
ASAPIGSAIPRNNWAVTCDSAQSGNECNKAIDGNKDTFWHTFYGANGDPKPPHTYTIDMKTTQNVSGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPVAAAIEPTSGRVLMWSSYRNDRFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSAKTWTFLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPHFEDSDATTNAHIITLGEPGTSPNTVFASNGLYFARTYHTSVVLPDGSTFITGGQRRGIPFEDSTPVFTPEIYVPEQDTFYKLNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

(19) United States
(12) Patent Application Publication
(10) Pub. No.: US 2026/0286044 A1
(43) Pub. Date: Sep. 24, 2026

(54) BIOBASED POLYESTERS AND METHODS OF MAKING AND USING SAME

(71) Applicant: SOLUGEN, INC., Houston, TX (US)

(72) Inventors: Camille Boucher-Jacobs, Houston, TX (US); Toni M. Lee, Missouri City, TX (US); Amit Desai, Sugar Land, TX (US); David Weiner, San Diego, CA (US); Sean Hunt, Houston, TX (US); Gaurab Chakrabarti, Houston, TX (US); Nicholas Sparkman, Pearland, TX (US)

(73) Assignee: Solugen, Inc., Houston, TX (US)

(21) Appl. No.: 19/476,936

(22) PCT Filed: Apr. 29, 2024
(86) PCT No.: PCT/US2024/026887

§ 371 Date: Oct. 20, 2025

Related U.S. Application Data
(60) Provisional application No. 63/498,735, filed on Apr. 27, 2023.

(51) Int. Cl. C08G 63/664; C07D 309/38; C07D 317/32; C07D 407/14; C07D 493/04; C08G 63/08

(52) U.S. Cl. CPC C08G 63/664; C07D 309/38; C07D 317/32; C07D 407/14; C07D 493/04; C08G 63/08

(57) ABSTRACT

A method for formation of a biobased monomer comprising contacting a sugar with one or more oxidation catalysts at least once under conditions suitable for the formation of a primary oxidation product; and subjecting the primary oxidation product to at least one process selected from the group consisting of oxidation, dehydration, acetylation, hydrogenation, ion exchange, and combinations thereof under conditions suitable for formation of a product mixture. A method for formation of a biobased monomer comprising contacting glucose with one or more oxidation catalysts at least once under conditions suitable for the formation of a primary oxidation product; and subjecting the primary oxidation product to at least one process selected from the group consisting of oxidation, dehydration, acetylation, hydrogenation, ion exchange, and combinations thereof under conditions suitable for formation of a product mixture.

### CROSS-REFERENCE TO RELATED APPLICATIONS

This application is a 35 U.S.C. § 371 national stage application of PCT/US2024/026887 filed Apr. 29, 2024, and entitled “Biobased Polyesters and Methods of Making and Using Same,” which claims priority to U.S. Provisional Application No. 63/498,735 filed Apr. 27, 2023 and entitled “Biobased Polymers and Methods of Making and Using Same,” each of which is hereby incorporated herein by reference in its entirety for all purposes.

### REFERENCE TO SEQUENCE LISTING

The instant application contains a Sequence Listing which has been submitted electronically in XML file format and is hereby incorporated by reference in its entirety. Said XML file, created on Apr. 29, 2024, is named “3416-10702_20PRC010PCT.xml” and is 2.45 kilobytes in size.

### FIELD

The present disclosure relates generally to polymer compositions and methods of making same. More particularly, this disclosure relates to methods for the production of sugar-based monomers and polymerization of these monomers to form polymeric materials.

### BACKGROUND

The worldwide interest in biobased polymers has accelerated in recent years due to the desire and need to find non-fossil fuel-based polymers. Although the terms “biobased polymers” and “biodegradable polymers” are sometime used interchangeably, there is a key difference between the two types of polymers. In particular, biobased polymers are materials produced from renewable resources and “biodegradable polymers” are defined as materials whose physical and chemical properties undergo deterioration and completely degrade when exposed to microorganisms, carbon dioxide (aerobic) processes, methane (anaerobic processes), and/or water (aerobic and anaerobic processes). Bio-based polymers can be biodegradable (e.g., polylactic acid) or nondegradable (e.g., biopolyethylene).

The synthesis of polymers such as polyesters typically employ harsh reaction conditions and environmentally unfriendly compounds such as organic solvents. In many petroleum-based polymers, specific structural properties such as higher glass transition temperature (Tg), greater ductility, changes in crystallization rate, enhanced material and structural elasticity are often achieved through the use of additives that may be toxic (e.g., phthalate plasticizers such as Bisphenol A or brominated flame retardants). Conventional biobased alternatives to petroleum-based polymers often suffer from poor material properties or high costs of production. An ongoing need exists for the development of biobased polymers with desirable material properties.

### BRIEF DESCRIPTION OF THE DRAWINGS

For a detailed description of various exemplary aspects, reference will now be made to the accompanying drawings in which:

FIG. 1 is a schematic of the reaction for production of glucaric and gluconic acid.

FIG. 2 is a schematic of the reaction of glucose to produce glucaric acid.

FIG. 3 is a schematic of the reaction for the production of D-gluconic acid acetate lactone.

FIG. 4 is a schematic of the reaction for chemoenzymatic synthesis of D-glucaric acid 1,4:6,3-dilactone from D-glucose.

FIG. 5 is a schematic of the reaction for chemoenzymatic synthesis of diisopropylidene D-glucaric acid methyl ester from D-glucose.

FIG. 6 is a schematic of the reaction for chemoenzymatic synthesis of diisopropylidene gluconic acid lactic acid lactone from D-glucose.

FIGS. 7A and 7B depict exemplary structures of polymers that may be produced from biobased monomers of the present disclosure.

FIG. 8 are an NMR spectra of D-gluconic acid acetate pyranone.

FIG. 9 is an NMR spectra of methyl diisopropylidene gluconic acid lactone produced from gluconolactone.

FIG. 10 depicts the NMR spectra of D-glucaric acid 1,4:6,3-dilactone product synthesized from calcium glucarate.

FIG. 11 is the NMR spectra of the product of dimethoxypropane reaction with glucaric acid.

### SUMMARY

A method for formation of a bioderived monomer comprising contacting a sugar with one or more oxidation catalysts at least once under conditions suitable for the formation of a primary oxidation product; and subjecting the primary oxidation product to at least one process selected from the group consisting of oxidation, dehydration, acetylation, hydrogenation, ion exchange, and combinations thereof under conditions suitable for formation of a product mixture.

A method comprising contacting a diisopropylidene gluconic acid α-hydroxy acid lactone with a ring opening polymerization catalyst under conditions suitable for the formation of a gluconic acid-derived poly(α-hydroxy acid)-type polyester.

A method for formation of a biobased monomer comprising contacting glucose with one or more oxidation catalysts at least once under conditions suitable for the formation of a primary oxidation product; and subjecting the primary oxidation product to at least one process selected from the group consisting of oxidation, dehydration, acetylation, hydrogenation, ion exchange, and combinations thereof under conditions suitable for formation of a product mixture.

### DETAILED DESCRIPTION

Disclosed herein are biobased compounds that may function as monomers, crosslinkers, property-modifying additives and the like. For example, in some aspects disclosed herein, the biobased compound is a monomer which can be polymerized to form homopolymers or copolymers. Also disclosed herein, in some aspects, are homopolymers and/or copolymers comprising a monomeric biobased compound.

The biobased compounds may be derived from or the oxidation of a sugar. In one or more aspects, the biobased monomer (BBM) is the product of multiple oxidation steps. In one or more aspects, a sugar is subjected to a first oxidation to produce a primary oxidation product (POP); additionally, in some aspects, the POP may be subjected to a second oxidation and the product termed a secondary oxidation product (SOP); additionally, in some aspects, the SOP subjected to a third oxidation and the product termed a tertiary oxidation product (TOP). In various aspects, any compound referred to as a POP, SOP or TOP may be obtained from a commercial source or may be prepared using a suitable oxidation catalyst. Nonlimiting examples of suitable oxidation catalysts are disclosed herein. In other aspects, a POP, SOP or TOP may be a reactant in other processes such as through hydrogenation, acetylation and dehydration to result in a product that functions as a BBM.

In an aspect, the BBM is a product derived from the oxidation of glucose. In such aspects, the BBM comprises gluconolactone (GDL), glucodialdose (GDA), glucaric acid (GA), or combinations thereof. Collectively GDL, GDA and GA are termed the primary oxidized products (POPs).

In one or more aspects, a sugar (e.g., glucose) is oxidized to form a POP. In one or more aspects, the POP is derivatized to form compounds such as D-gluconic acid acetate lactone (GO-OAcL), D-glucaric acid 1,4:6,3-dilactone (GAdL), diisopropylidene D-gluconic acid lactic acid lactone (DIP-GO-LAL), and diisopropylidene D-glucaric acid (DIP-GA). Collectively these sugar derivatives are termed biobased monomers (BBM).

In some aspects, one or more BBMs may be used in the production of biobased polymers, herein collectively termed (BBP). In one of more aspects, BBMs function as a monomeric compound that, under suitable conditions, form polymeric molecules displaying desirable mechanical and/or physical properties such as a high glass transition temperature, increased tensile strength, improved Young's modulus, corrosion resistance, permeability, enhanced biodegradability or combinations thereof.

In one or more aspects, the reactions disclosed herein comprising a biocatalyst may be carried out under conditions suitable for the formation of the desired product. In one or more aspects, the reactions disclosed herein comprising a metal catalyst (e.g., transition metal catalyst, supported metal catalyst) may be carried out under conditions suitable for the formation of the desired product.

A POP suitable for use in the present disclosure may comprise GDL which may be obtained from any source. For example, GDL may be synthesized by contacting glucose with a glucose oxidase catalyst (GOX) in the presence of oxygen under conditions suitable for the formation of GDL. Conditions suitable for the formation of GDL may include a GOX amount ranging from about 1 ppm to about 1000 ppm alternatively form about 1 ppm to about 100 ppm, a temperature of from about 0° C. to about 50° C. a reaction media pH ranging from about 4 to about 7 and a reaction time ranging from about 1 hour to about 72 hours, alternatively from about 3 hours to about 24 hours or alternatively from about 5 hours to about 10 hours. This reaction generates hydrogen peroxide as a by-product consequently, a catalase may be a component of the reaction media in order to disproportionate the hydrogen peroxide.

In one or more aspects, GDL is further processed or reacted to form additional downstream products. For example, GDL may be reacted with a strong base (e.g., NaOH) under conditions suitable for the formation of gluconic acid. Conditions suitable for the formation of gluconic acid include an amount of base that is stochiometric to the amount of gluconic acid and a temperature of from about 0 to about 50° C.

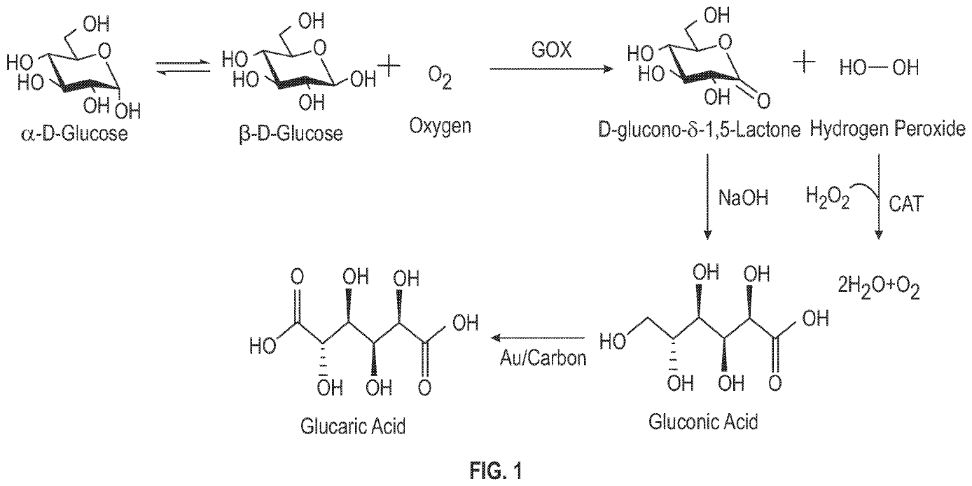

Additional oxidized products may be obtained by reacting gluconic acid with another oxidizing catalyst under conditions suitable for the formation of P glucaric acid. In one or more aspects, the oxidizing catalyst is a transition metal catalyst such as Au/Pt on carbon support. These reactions are depicted schematically in FIG. 1.

In an aspect, the POP comprises GDA which can be obtained from any suitable source. For example, GDA can be synthesized by first reacting glucose with a biocatalyst (e.g., an oxidase). In one or more aspects, the catalyst is galactose oxidase or a mutant thereof. In such aspects, the reaction may be carried out in the presence of a small molecule activator such as horseradish peroxide. This reaction generates hydrogen peroxide as a by-product consequently a catalase may be a component of the reaction media in order to disproportionate hydrogen peroxide.

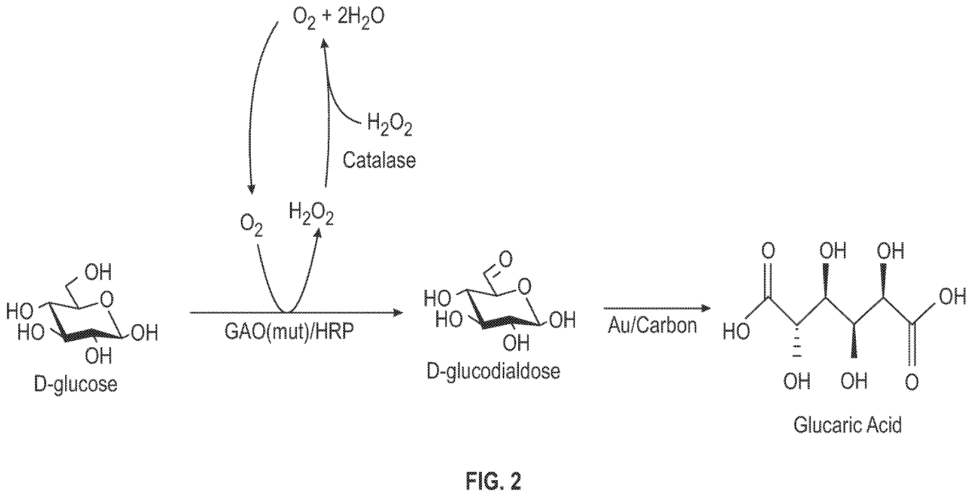

In one or more aspects, GDA is converted to gluconic acid (e.g., a SOP) by reaction with a suitable oxidizing catalyst. In one or more aspects, the oxidizing catalyst is a transition metal catalyst such as gold or platinum on a carbon support material. These reactions are depicted schematically in FIG. 2.

In one or more aspects, the POP is D-gluconic acid-lactone (D-GO-lactone). D-GO-lactone may be formed by the oxidation of glucose by a GOX under suitable conditions. In one or more aspects, D-GO-lactone is used to form the SOP gluconic acid acetate pyranone (GO-OAc-pyranone). For example, D-GO-lactone may be reacted with acetate and pyridine under suitable conditions. Conditions suitable for the formation of GO-OAc-pyranone include an amount of D-GO-lactone an amount of acetate ranging from about 4 equivalents to about 20 equivalents and an amount of pyridine ranging from about 4 equivalents to about 20 equivalents.

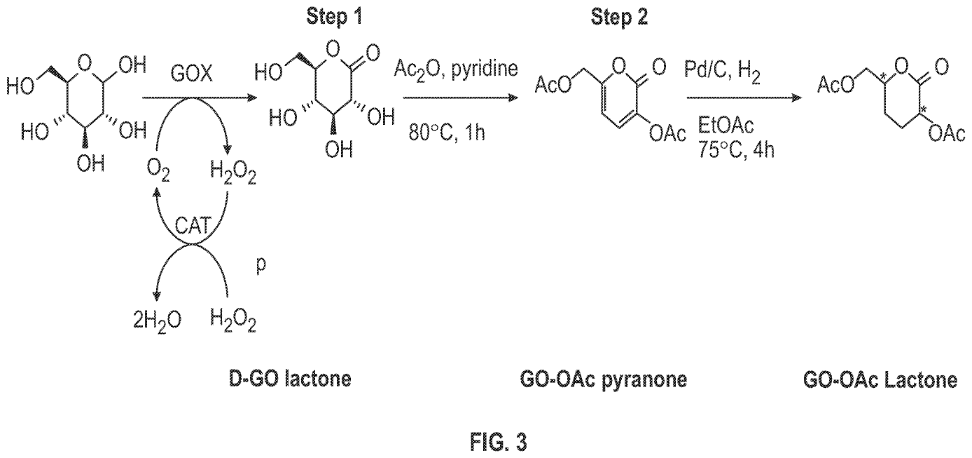

In an aspect, the method further comprises hydrogenation of GO-OAc-pyranone in the presence of a hydrogenation catalyst under conditions suitable for the formation of D-GO-lactone. Conditions suitable for the formation of D-GO-lactone include an amount of GO-OAc-pyranone and an amount of hydrogenation catalyst each ranging from about 5 wt. % to about 50 wt. % based on total weight of the reaction mixture, a temperature ranging from about 15° C. to about 60° C. and a reaction time ranging from about 2 hrs to about 24 hrs. A suitable hydrogenation catalyst may contain from about 5 weight percent (wt. %) to 10 wt. % of Pd on carbon based on the total weight of the catalyst. These hydrogenation reactions of GO-OAc-pyranone are depicted schematically in FIG. 3.

In one or more aspects, the BBM is 1,4:6,3 glucaric acid dilactone (GAdL) which may be obtained from any suitable source. For example, the GAdL is prepared by the oxidation of glucose to the corresponding dialdose (a POP) in the presence of an oxidase. In one or more aspects, the catalyst is galactose oxidase or a mutant thereof. In such aspects, the reaction is carried out in the presence of a small molecule activator such as horseradish peroxide. This reaction generates hydrogen peroxide as a by-product consequently a catalase may be a component of the reaction media in order to disproportionate the hydrogen peroxide.

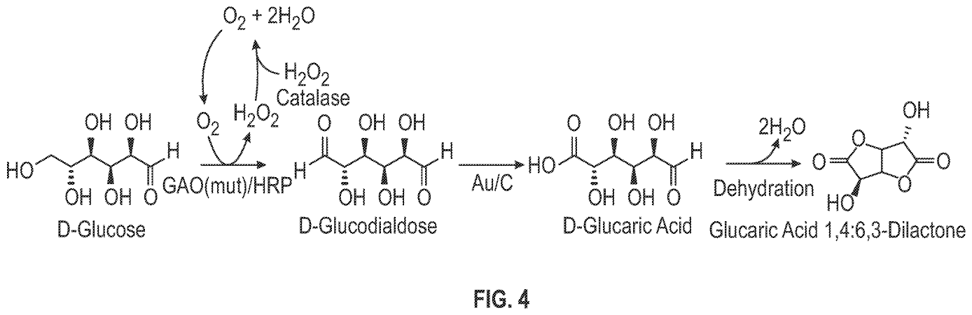

The dialdose may then be oxidized in the presence of a transition metal oxidation catalyst to form the SOP, glucaric acid, which is subsequently dehydrated to form glucaric acid 1,4:6,3-dilactone. Oxidation of the dialdose may be carried out using a transition metal oxidation catalyst (e.g., Au/Pt/carbon). Dehydration of glucaric acid may be carried out using a dehydration catalyst such as a sulfuric acid in a reaction solvent such as methyl isobutyl ketone at temperatures ranging from about 90° C. to about 120° C. for a reaction time of from about 6 hrs to about 24 hrs. Reactions for the oxidation of the dialdose are depicted schematically in FIG. 4.

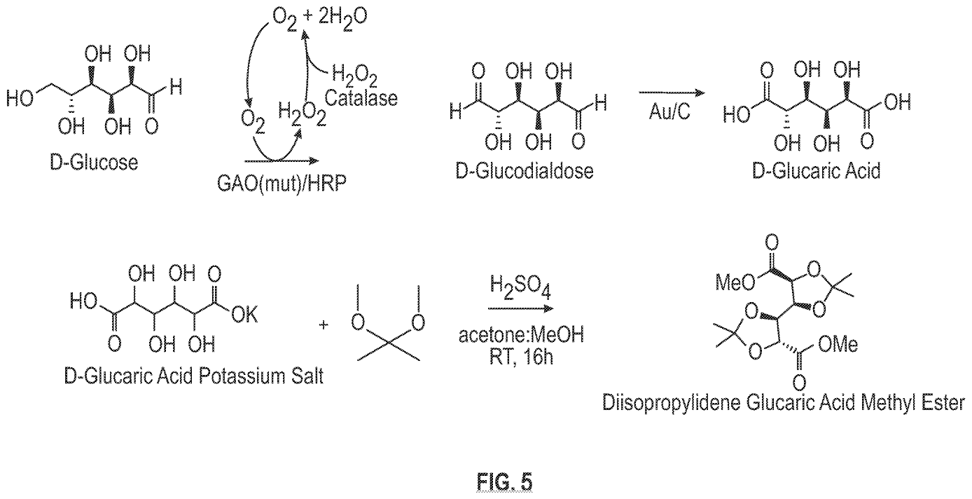

In one or more aspects, the BBM is diisopropylidene glucaric acid methyl ester. Diisopropylidene glucaric acid methyl ester may be formed from a salt of glucaric acid (e.g., potassium or calcium) which is reacted with an alkoxy-containing compound under conditions suitable for the formation of the diisopropylidene glucaric acid methyl ester. Conditions suitable for the formation of the diisopropylidene glucaric acid methyl ester may comprise an amount of alkoxy containing compounds ranging from about 4 equivalents to about 10 molar equivalents per molar equivalent of the glucaric acid salt, a solvent such as a mixture of methanol and acetone, a temperature of from about 10° C. to about 30° C. and a reaction time of from about 12 hours to about 48 hours. The reaction is depicted schematically in FIG. 5. In one or more aspects, a BBM is polymerized to form a BBP.

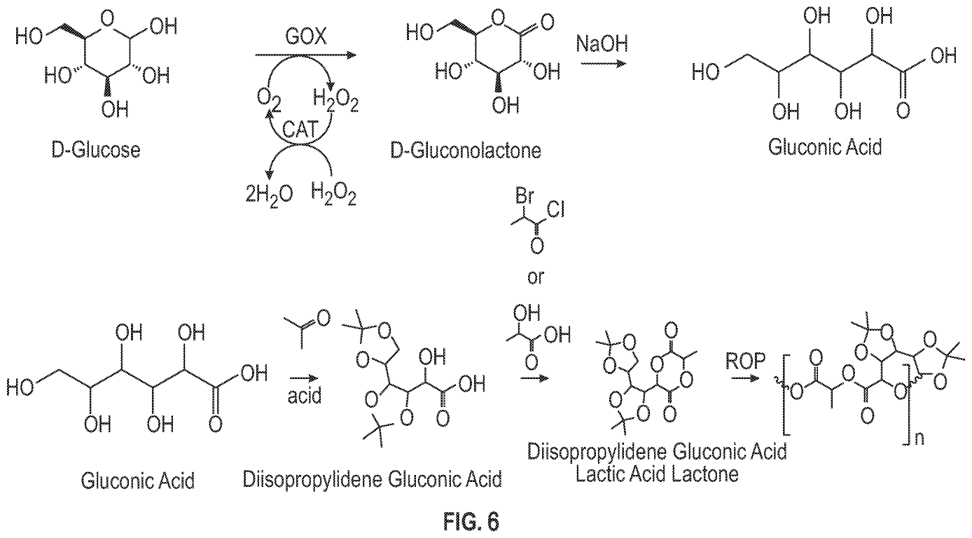

In one or more aspects, the BBP comprises a gluconic acid-derived poly(α-hydroxy acid)-type polyester such as DIP-GO-LAL. A scheme for formation of DIP-GO-LAL from gluconic acid is depicted in FIG. 6. With reference to FIG. 6, gluconic acid, derived from glucose is reacted with an acid catalyst and a ketone to form diisopropylidene gluconic acid. In one or more aspects, the ketone comprises acetone, ethyl methyl ketone, methyl isobutyl ketone, or combinations thereof. In one or more aspects, the ketone comprises acetone. The acid catalyst may comprise a sulfuric acid. The reaction may be carried out using the ketone as a solvent, an acid catalyst present in an amount of from about 2 wt. % to about 25 wt. % at a temperature of from about 15° C. to about 30° C. and a reaction time of from about 6 hours to about 48 hrs. The weight percent is based on the total weight of the reaction mixture.

In one or more aspects, diisopropylidene gluconic acid is then reacted with an α-hydroxy acid to form a diisopropylidene gluconic acid α-hydroxy acid lactone. Any α-hydroxy acid may be reacted with diisopropylidene gluconic acid. Nonlimiting aspects of α-hydroxy acid suitable for use in the present disclosure include lactic acid, glycolic acid, malic acid, citric acid, tartaric acid and combinations thereof. In one or more aspects, the α-hydroxy acid comprises lactic acid. The reaction may be carried out using a solvent, diisopropylidene gluconic acid present α-hydroxy acid present in an amount of from about 1 equivalent to about 2 equivalents based on the number of equivalents of diisopropylidene gluconic acid at a temperature of from about 0° C. to about 60° C. and for a reaction time of from about 1 hr to about 24 hrs.

The diisopropylidene gluconic acid α-hydroxy acid lactone may be subjected to ring-opening polymerization (ROP). ROP is a form of chain-growth polymerization in which the terminus of a polymer chain attacks cyclic monomers to form a longer polymer. The ROP may be carried out using any suitable mechanism such anionic ring-opening polymerization, cationic ring-opening polymerization or ring-opening metathesis polymerization using a suitable ROP catalyst such as aluminum complexes, guanidine metal complexes, for example, Na₂SnO₃ and the like. The resulting polymerized material, designated BIOPOL, may be reacted with another designated BIOPOL1 or with a different monomer to form a larger polymer or copolymer designated (BIPOL1)ₙ where n ranges from about 5 to about 70.

In another aspect, any BBM of the present disclosure may function as a monomer to produce a polymeric compound (e.g., copolymer, terpolymer) having n differing monomers where n ranges from 1 to 10, additionally or alternatively, from 1 to 5. In some aspects, the BBM may be a comonomer that is included with any other suitable monomer to form a polymer having any suitable structure such as a linear copolymer, grafted copolymer, block copolymer, or the like. For example, the BBM may form a copolymer with monomers such as ethylene, propylene, styrene, tetrafluoroethylene, vinyl chloride, lactic acid, phenol-formaldehyde, or acrylonitrile.

In another aspect, a BBM of the present disclosure may function as a crosslinker to enhance the properties of a polymeric material. Herein, a crosslinker refers to an additive that links two polymer chains by a covalent or ionic bond.

In some aspects, an enzyme utilized in the production of a BBM is a member of the copper radical oxidase family. For example, and without limitation, a copper radical oxidase suitable for use in the present disclosure is galactose oxidase (GAO, EC 1.1. 3.9). Other members in the copper radical oxidase family may be suitably employed in the present disclosure.

GAO is a copper-dependent alcohol oxidase that oxidizes galactose residues either as monosaccharides or glycoconjugates that contain galactose at the nonreducing end. GAO is a novel metallo-radical complex comprising a protein radical coordinated to a copper ion in the active site. The unusually stable protein radical is formed from the redox-active side chain of a cross-linked tyrosine residue (Tyr-Cys). In one or more aspects, the GAO is mutated to allow for the use of a glucose substrate and may have SEQ ID NO1. In one or more aspects, the GAO reaction is carried out in the presence of a small molecule activator. The small molecule activator is a cofactor that functions to rescue the activity of the GAO. Nonlimiting examples of other small molecule activators suitable for use in the present disclosure include L-tryptophan, 2-mercaptobenzothiazole, L-histidine, methylchloroisothiazolinone, o-dianisidine, 2,2′-azino-bis(3-ethylbenzothiazoline-6-sulfonic acid (ABTS), 4-aminoantipyrine, L-tyrosine, (2,2,6,6-tetramethylpiperidin-1-yl)oxyl, chloromethylisothiazolinone, 4-thiazolecarboxylic acid, Sunset yellow FCF, tartrazine, p-benzoquinone, dicoumarol, phthalimide, saccharin, phthalic anhydride, erythrosine B, 2-aminobenzothiazole, thiabendazole, 2-hydroxybenzothiazole, phenothiazine, 6-aminobenzothiazole, indigo carmine, naphthalimide, 2-aminothiazole, thiazole, 2H-1,4-benzothiazin-3(4H)-one, 2-oxindole, beta-lapachone, menaquinone, thiamine, 4-methyl-5-thiazoleethanol, Allura Red AC, menadione, p-cresol, Fast green FCF, Brilliant Blue FCF, methylisothiazolinone, caffeine, veratryl alcohol, fluorescein, and combinations thereof.

In an aspect, an enzyme utilized in the production of a BBM is a glucose oxidase. Glucose oxidase (EC number 1.1.3.4, herein “GOX”) is a soluble, homodimeric, secreted flavoprotein that oxidizes β-D-glucose (a natural isomerization product in equilibrium with α-D-glucose) to D-glucono-δ-1,5-lactone while reducing molecular oxygen to form hydrogen peroxide. GOX is commercially available for many uses including the determination of free glucose in sera or blood plasma for diagnostics, as a monitoring agent in fermentation processes, for controlling glucose in vegetal raw material and food products, as an additive in baked goods or egg products, and as an oxygen removal agent in packaged foods.

As will be understood by one of ordinary skill in the art with the benefit of the present disclosure, reactions of the type disclosed herein (e.g., enzyme oxidation of glucose) result in the production of hydrogen peroxide (H₂O₂) that can detrimentally impact other components of the reaction mixture. Specifically, the H₂O₂ produced may adversely affect the enzyme activity and structure. BBMs of the type disclosed herein may be produced in the presence of catalase (E.C. 1.11.1.61) which catalyzes the disproportionation of H₂O₂ to form H₂O and O₂, thus mitigating the detrimental effects of hydrogen peroxide on the enzymes present.

In an aspect, a catalyst for use in the present disclosure comprises a supported transition-metal oxidation catalyst. In an aspect, the support comprises carbon, silica, alumina, titania (TiO₂), zirconia (ZrO₂), a zeolite, or any combination thereof, which contains less than about 1.0 weight percent (wt. %), alternatively less than about 0.1 wt. % or alternatively less than about 0.01 wt. % SiO₂ binders based on the total weight of the support.

Suitable support materials are predominantly mesoporous or macroporous, and substantially free from micropores. For example, the support may comprise less than about 20% micropores. In an aspect, the support is a porous nanoparticle support. As used herein, the term “micropore” refers to pores with diameter less than 2 nm, as measured by nitrogen adsorption and mercury porosimetry methods and as defined by IUPAC. As used herein, the term “mesopore” refers to pores with diameter from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In some aspects, the support comprises a mesoporous carbon extrudate having a mean pore diameter ranging from about 10 nm to about 100 nm and a surface area greater than about 20 m² g⁻¹ but less than about 300 m² g⁻¹. Supports suitable for use in the present disclosure may have any suitable shape. For example, the support may be shaped into 0.8-3 mm trilobes, quadralobes, or pellet extrudates. Such shaped supports enable the used of fixed trickle bed reactors to perform the final oxidation step under continuous flow.

In one or more aspects, the metal comprises a Group 8 metal (e.g., Re, Os, Ir, Pt, Ru, Rh, Pd, Ag), a 3d transition metal, an early transition metal, or combinations thereof. In an aspect, the transition metal catalyst comprises gold, Au, alternatively the transition metal catalyst comprises platinum, Pt.

In an aspect, the transition metal catalysts are heterogeneous, solid-phase catalysts. In such aspects, the catalyst support may be treated so as to promote the preferential deposition of the metal (e.g., Au) on the outer surface of the support so as to create a shell type catalyst. In general, the transition metal catalyst may be prepared using any suitable methodology. For example, the transition metal catalyst may be prepared using incipient wetness impregnation, ion-exchange and deposition-precipitation.

In an aspect, the BBMs of the present disclosure (e.g., lactones such as DIP-GO-LAL, GAdL, GO-OAcL) may be polymerized using ROP reaction conditions. BBMs of the present disclosure may be formed into homopolymers or copolymers such as copolymers with other lactones such as lactide, caprolactone or valerolactone.

In one or more aspects, the BBMs of the present disclosure are contacted with a transesterification catalyst, a polycondensation catalyst, and a diol (such as ethylene glycol and other C₂-C₆ diols) under conditions suitable for the formation of a polymer. In other aspects, BBMs of the present disclosure are copolymerized with commercially available diacids (such as succinic acid). In yet other aspects, a BBM such as GAdL may function as a diol in polyesters structures with materials such as diacids and/or dicarboxylic acids.

In an aspect, polymerization of the BBM to form a BBP is carried out in the presence of a suitable catalyst. Nonlimiting examples of catalysts suitable for use in the present disclosure include Bu₂SnO with cyclic SnO, Zn(OAc)₂ with Sb₂O₃, Ti(isopropoxide)₄ with Ti(butoxide)₄ and/or Sb₂O₃, Sb₂O₃, Co(OAc)₂, 1,8-diazabicyclo[5.4.0]undec-7-ene (DBU), Ti(butoxide)₄, Al(acac)₃, Zn(OAc)₂, Na and Mg (elemental), “metal oxides” of Sn, Pb, Ti or Hf, Ti catalyst by itself or with Mg, Sc(OTf)₃, or combinations thereof.

In one or more aspects, the ratio of catalyst to monomer for any polymerization reaction disclosed herein may range from about 1:50 to about 1:600 equivalent, additionally or alternatively from about 1:20 to about 1:50 or, additionally or alternatively, from about 1:600 to about 1:1000 at a temperature of from about 90° C. to about 150° C., additionally or alternatively, from about 10° C. to about 50° C. or from about 0° C. to a bout 25° C., for a reaction time of from about 1 minute to about 10 hours, additionally or alternatively, from about 1 hr to about 12 hrs, additionally or alternatively, from about 10 min to about 1 hr in a suitable solvent.

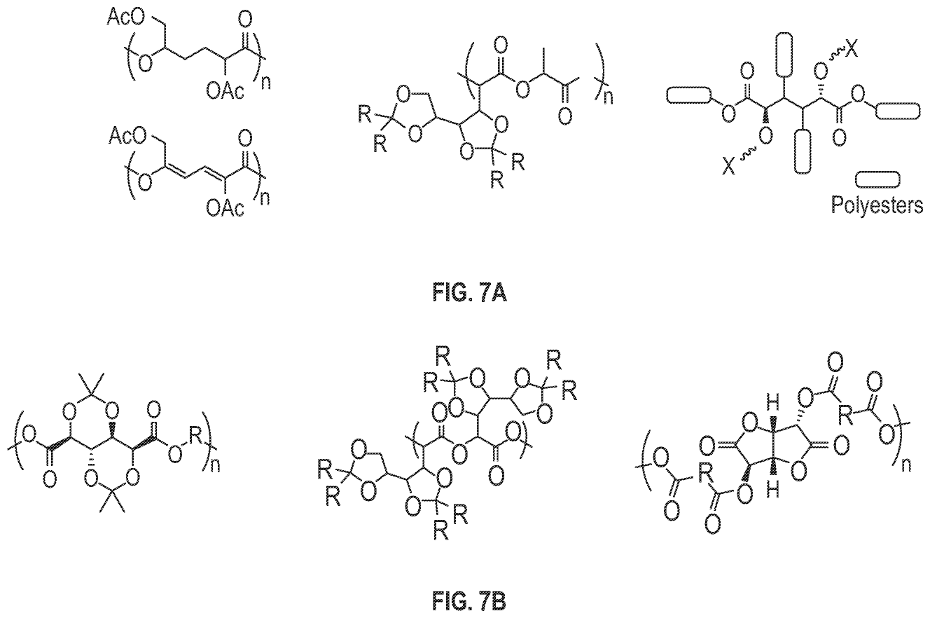

Nonlimiting examples of a suitable solvent include ethers, carbonates, esters, ketones, aldehydes, or alcohols. Suitable ether solvents can be cyclic or acyclic. Nonlimiting examples of suitable ethers which can be useful as a solvent include dimethyl ether, diethyl ether, methyl ethyl ether, monoethers or diethers of glycols (e.g., dimethyl glycol ether), furans, substituted furans, dihydrofuran, substituted dihydrofurans, tetrahydrofuran (THF), substituted tetrahydrofurans, tetrahydropyrans, substituted tetrahydropyrans, 1,3-dioxanes, substituted 1,3-dioxanes, 1,4-dioxanes, substituted 1,4-dioxanes, or mixtures thereof. Non-limiting examples of suitable carbonates which can be utilized as a solvent include ethylene carbonate, propylene carbonate, diethyl carbonate, diethyl carbonate, glycerol carbonate, and combinations thereof. Non-limiting examples of suitable esters which can be utilized as a solvent include ethyl acetate, propyl acetate, butyl acetate, isobutyl isobutyrate, methyl lactate, ethyl lactate, and combinations thereof. Non-limiting examples of suitable ketones which can be utilized as a solvent include acetone, ethyl methyl ketone, methyl isobutyl ketone, and combinations thereof. Non-limiting examples of suitable alcohols which can be utilized as a solvent include methanol, ethanol, propanol, isopropanol, n-butanol, isobutanol, pentanol, hexanol, heptanol, octanol, benzyl alcohol, phenol, cyclohexanol, and the like, or combinations thereof. Nonlimiting example structures of BBPs of the type disclosed herein are presented in FIGS. 7A and 7B.

In an aspect, the BBM is GO-OAcL. GO-OAcL is a monomer that could replace lactic acid based polymers generated through ROP. ROP of functionalized lactones is appealing due to the ability to tightly control the reaction. Polylactide lacks chemical functional groups leading to high hydrophobicity and slow degradation times. Lactones originating from sugars, however, should contain the requisite functional groups to generate polymers that are both biodegradable and exhibit lower hydrophobicity.

GAdL is a molecule that could be useful not only as a monomer for the synthesis of polymers, but also the generation of protein-resistant materials, and nucleic acid delivery agents. Aldarodilactones such as GAdL are particularly useful as acylating agents and crosslinkers as they are more reactive than their corresponding diesters and generate no byproducts during ester or amide formation. GAdL is a crystalline solid that can be stored indefinitely at room temperature and dispensed in known stoichiometric amounts.

DIP-GO-LAL monomer may form poly(α-hydroxy acid)-type polyesters through ROP). Formation of the diisopropylidene form of gluconic acid through reacting with acetone may be effective to protect the 3, 4, 5, and 6-hydroxyl groups before lactonization with an alpha-hydroxy acid such as lactic acid and polymerization with itself or as a copolymer with DL-lactide or another lactone cyclic ester. The resulting structure (e.g., a DIP-GO-LAL monomer) can then be polymerized via ROP either with itself or another monomer.

Any oxidation product of the present disclosure (e.g., POP,SOP,TOP) may be produced in yields of from about 40% to about 95%, alternatively from about 50% to about 95% or alternatively from about 80% to about 95%

The BBPs can be processed using existing polymer-processing equipment and can be converted into injection-molded components: film and sheet, fibers, laminates, and coated articles; nonwoven fabrics, synthetic paper products, disposable items, feminine hygiene products, adhesives, waxes, paints, binders, and foams. BBPs are suitable for a wide range of food packing applications including caps and closures, disposable items such as forks, spoons, knives, tubs, trays, and hot cup lids, and products such as housewares, cosmetics, and medical packaging.

The BBMs of the present disclosure may be polymerized to obtain materials that are used in areas such as the production of woven fibers (e.g., carpets, textile, molded parts, clothes and recycling); the production of construction fibers (e.g., fiberglass replacement, carbon fiber replacement, mineral wool); and the production of rigid and flexible packaging (e.g., beverage bottles, thermoformed storage boxes). Other applications include the use of these materials as insulation (e.g., automotive heat insulation, electroinsulation, pipes); as polymer additives (e.g., plasticizer, anti-plasticizer); and as property-modifying additives (e.g., improve mechanical strength, thermal stability, moisture absorption, chemical resistance in plastics).

### ADDITIONAL DISCLOSURE

A first aspect which is a method for formation of the biobased monomer comprising: contacting a sugar with one or more with one or more oxidation catalysts at least once under conditions suitable for the formation of a primary oxidation product; and subjecting the primary oxidation product at least one process selected from the group consisting of oxidation, dehydration, acetylation, hydrogenation, ion exchange, and combinations thereof under conditions suitable for formation of a product mixture.

A second aspect which is the method of the first aspect wherein the sugar comprises glucose.

A third aspect which is the method of any of the first through second aspect wherein the primary oxidation product comprises D-gluconic acid lactone.

A fourth aspect which is the method of any of the first through third aspects wherein the at least one process comprises acetylation and the product mixture comprises D-gluconic acid pyranone.

A fifth aspect which is the method of any of the first through fourth aspects wherein the primary oxidation product comprises glucaric acid.

A sixth aspect which the method of any of the second through fifth aspects wherein the at least one process comprises oxidation with a transition metal oxidation catalyst and the product mixture comprises 1,4:6,3 glucaric acid dilactone.

A seventh aspect which is the method of any of the second through sixth aspects wherein the primary oxidation product comprises gluconic acid.

An eighth aspect which is the method of any of the first through seventh aspects further comprising isolating at least one monomer from the product mixture.

A ninth aspect which is the method of the eighth aspect further comprising polymerizing the at least one monomer to form a biobased polymer.

A tenth aspect which is the method of the ninth aspect wherein the polymerization is carried out in the presence of a comonomer to form a copolymer.

An eleventh aspect which is the method of the tenth aspect wherein the copolymer comprises a polyester.

A twelfth aspect which is the method of any of the first through eleventh aspects wherein the oxidation catalyst comprises an oxidase.

A thirteenth aspect which is the method of the twelfth aspect wherein the oxidase comprises galactose oxidase.

A fourteenth aspect which is the method of the first through thirteenth aspects wherein the oxidation catalyst comprises a transition metal.

A fifteenth aspect which is the method of the fourteenth aspect wherein the transition metal comprises Re, Os, Ir, Pt, Ru, Rh, Pd, Ag, Au, or combinations thereof.

A sixteenth aspect which is a method comprising contacting a diisopropylidene gluconic acid α-hydroxy acid lactone with a ring opening polymerization catalyst under conditions suitable for the formation of a gluconic acid-derived poly(α-hydroxy acid)-type polyester.

A seventeenth aspect which is the method of the sixteenth aspect wherein the α-hydroxy acid comprises lactic acid, glycolic acid, malic acid, citric acid, tartaric acid and combinations thereof.

An eighteenth aspect which is a method for formation of a biobased monomer comprising contacting glucose with one or more with one or more oxidation catalysts at least once under conditions suitable for the formation of a primary oxidation product; and subjecting the primary oxidation product at least one process selected from the group consisting of oxidation, dehydration, acetylation, hydrogenation, ion exchange, and combinations thereof under conditions suitable for formation of a product mixture.

A nineteenth aspect which is the method of the eighteenth aspect wherein the primary oxidation product comprises gluconolactone (GDL), glucodialdose (GDA), glucaric acid (GA), or combinations thereof.

A twentieth aspect which is the method of any of the eighteenth or nineteenth aspects wherein the primary oxidation product is formed at a yield of from about 40% to about 95%.

A twenty-first aspect which is an extruded article formed from the polymeric material of any of the ninth through eleventh aspects.

A twenty-second aspect which is an extruded article formed from the polymeric material of any of the sixteenth through seventeenth aspects.

A twenty-third aspect which is an article comprising a fiber formed from the polymeric material of any of the ninth through eleventh aspects.

A twenty-fourth aspect which is a coating comprising the polymeric material of any of the sixteenth through seventeenth aspects.

A twenty-fifth aspect which is an adhesive comprising the polymeric material of any of the sixteenth through seventeenth aspects.

### EXAMPLES

The aspects having been generally described, the following examples are given as particular aspects of the disclosure and to demonstrate the practice and advantages thereof. It is understood that the examples are given by way of illustration and are not intended to limit the specification or the claims in any manner.

### Example 1

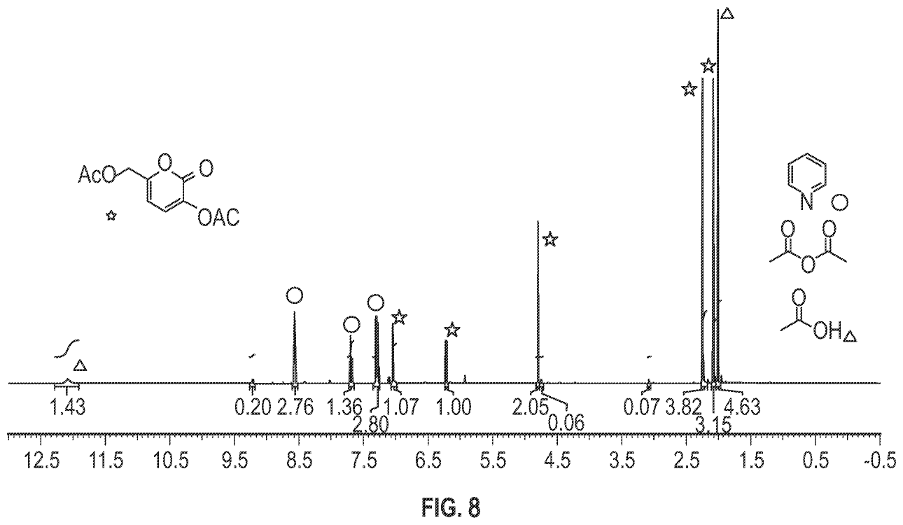

The synthesis of GO-OAc-pyranone was carried out. Specifically, 6 grams of D-GO lactone was reacted with 20 milliliters of acetic anhydride and pyridine at 80° C. for hour. The product mixture was analyzed by nuclear magnetic resonance (NMR) spectroscopy. The NMRspectra of the product mixture demonstrating the formation of GO-OAc-pyranone is presented in FIG. 8.

### Example 2

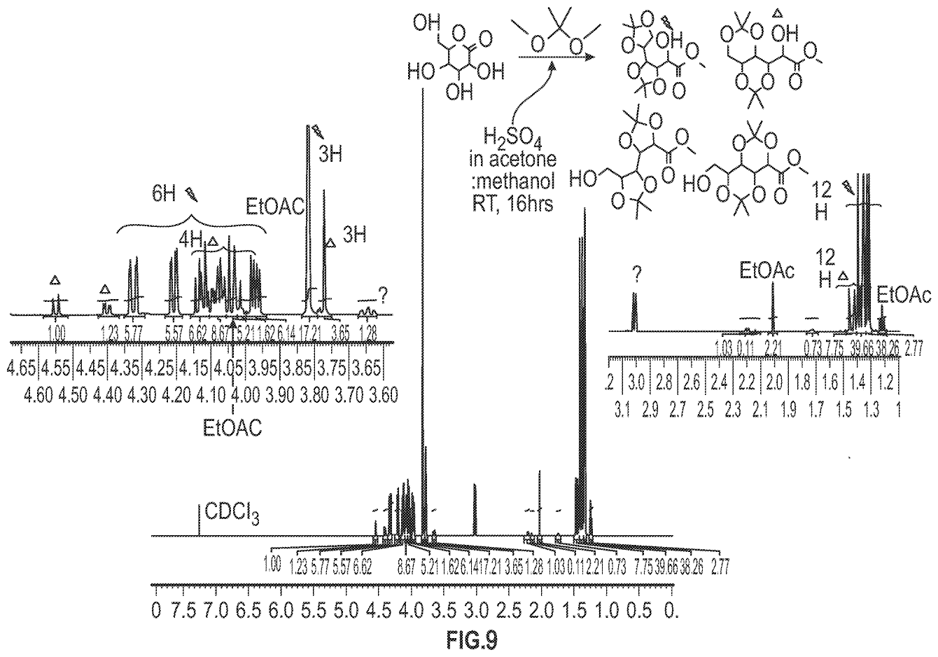

Glucaric acid dilactone was synthesized from calcium glucarate. Specifically, calcium glucarate was first refluxed with one equivalent of sulfuric acid in a 95:5 mixture of acetone:water for 4 hours at 80° C. The solution was filtered, and the volume adjusted with acetone. Next, a 1:1 volume of this solution to methyl isobutyl ketone (MIBK) was prepared, fractionally distilled (56-85° C.), refluxed at 115° C., 30 min, distilled to ⅓rd the volume (115-119° C.), and the product crystallized/recrystallized. The synthesis of GAdL was verified via NMR and the spectra is presented in FIG. 9.

### Example 3

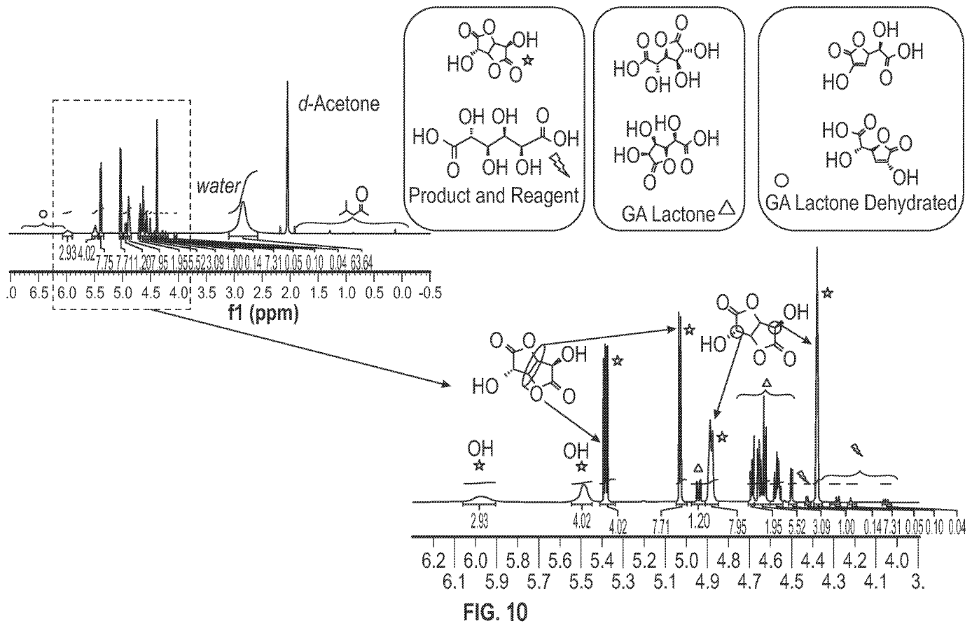

Synthesis of the diisopropylidene gluconic acid methyl ester (DIP-GA) precursor to DIP-GO-LAL was carried by reacting gluconolactone with acetone and dimethyoxypropane. Specifically, a vessel was charged with 4.14 g of gluconolactone in acetone:MeOH 1:1 50 mL dimethyoxypropane (17.15 mL) was added along with p toluene sulfonic acid (200 mg) substituted with H₂SO₄ (114 mg) and allowed to react for 16 hrs. The resulting product was analyzed with NMR and confirmed as the diisopropylidene gluconic acid methyl ester. The NMR spectra of the product mixture is presented in FIG. 10. DIP-GA compound was produced at a yield of 17%.

### Example 4

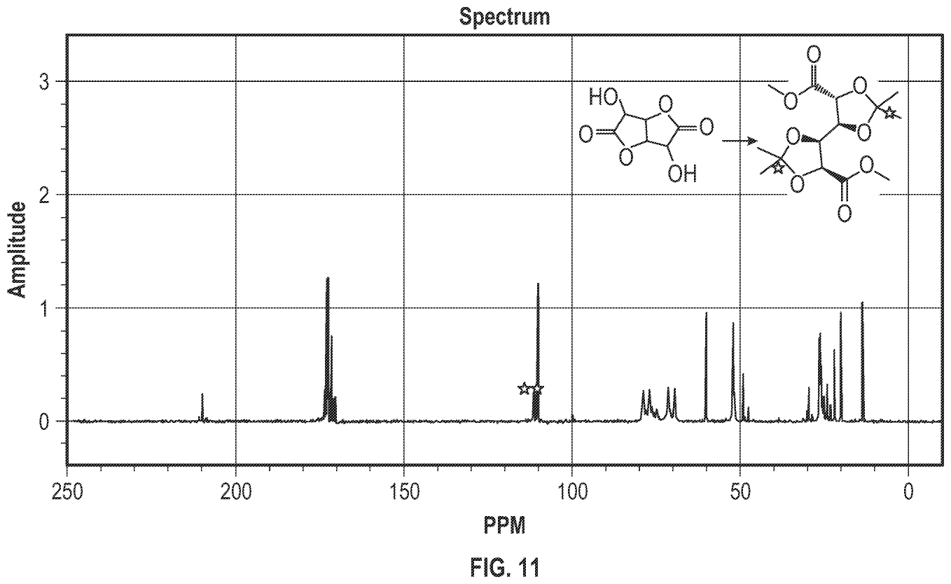

Synthesis of the diisopropylidene gluconic acid methyl ester (DIP-GA) precursor to DIP-GO-LAL was carried by reacting 25 mmol GAdL (1 eq of GAdL) with 6 equivalents of dimethoxypropane (150 mmol) in the presence of 10 mol % methanesulfonic acid (MSA) catalyst and 50 mL of 50-50 MeOH/acetone solvent. The reaction was carried out at room temperature for 27 hrs at which point it is washed with dilute cold bicarbonate and extracted into ethyl acetate. The reaction mass was rotovapped to dryness to afford a clear pale yellow oil. NMR spectroscopy was carried out to confirm installation of DIP group onto GA as well as presence of residual dimethoxypropane. The NMR spectra is presented in FIG. 11.

While aspects of the presently disclosed subject matter have been shown and described, modifications thereof can be made by one skilled in the art without departing from the spirit and teachings of the subject matter. The aspects described herein are exemplary only, and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the presently disclosed subject matter. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A method for formation of a biobased monomer, the method comprising:
contacting a sugar with one or more oxidation catalysts at least once under conditions suitable for the formation of a primary oxidation product; and
subjecting the primary oxidation product to at least one process selected from the group consisting of oxidation, dehydration, acetylation, hydrogenation, ion exchange, and combinations thereof under conditions suitable for formation of a product mixture.

2. The method of claim 1 wherein the sugar comprises glucose.

3. The method of claim 2, wherein the primary oxidation product comprises D-gluconic acid lactone.

4. The method of claim 3, wherein the at least one process comprises acetylation and the product mixture comprises D-gluconic acid pyranone.

5. The method of claim 2, wherein the primary oxidation product comprises glucaric acid.

6. The method of claim 3, wherein the at least one process comprises oxidation with a transition metal oxidation catalyst and the product mixture comprises 1,4:6,3 glucaric acid dilactone.

7. The method of claim 2, wherein the primary oxidation product comprises gluconic acid.

8. The method of claim 1, further comprising isolating at least one monomer from the product mixture.

9. The method of claim 8, further comprising polymerizing the at least one monomer to form a biobased polymer.

10. The method of claim 9, wherein the polymerization is carried out in the presence of a comonomer to form a copolymer.

11. The method of claim 10, wherein the copolymer comprises a polyester.

12. The method of claim 1, wherein the oxidation catalyst comprises an oxidase.

13. The method of claim 12, wherein the oxidase comprises galactose oxidase.

14. The method of claim 1, wherein the oxidation catalyst comprises a transition metal.

15. The method of claim 14, wherein the transition metal comprises Re, Os, Ir, Pt, Ru, Rh, Pd, Ag, Au, or combinations thereof.

16. A method, comprising:
contacting a diisopropylidene gluconic acid α-hydroxy acid lactone with a ring opening polymerization catalyst under conditions suitable for the formation of a gluconic acid-derived poly(α-hydroxy acid)-type polyester.

17. The method of claim 16, wherein the α-hydroxy acid comprises lactic acid, glycolic acid, malic acid, citric acid, tartaric acid and combinations thereof.

18. A method for formation of a biobased monomer, the method comprising:
contacting glucose with one or more oxidation catalysts at least once under conditions suitable for the formation of a primary oxidation product; and
subjecting the primary oxidation product to at least one process selected from the group consisting of oxidation, dehydration, acetylation, hydrogenation, ion exchange, and combinations thereof under conditions suitable for formation of a product mixture.

19. The method of claim 18, wherein the primary oxidation product comprises gluconolactone, glucodialdose, glucaric acid, or combinations thereof.

20. The method of claim 18, wherein the primary oxidation product is formed at a yield of from about 40% to about 95%.
